(19) United States
(12) Patent Application Publication
(10) Pub. No.: US 2023/0372468 A1
(43) Pub. Date: Nov. 23, 2023

(54) COMPOSITIONS AND METHODS FOR TARGETING CORONAVIRUS USING LIPID VESICLES INCLUDING EXOSOMES

(71) Applicant: Board of Regents, The University of Texas System

(72) Inventor: RAGHU KALLURI, Houston, TX (US)

(73) Assignee: Board of Regents, The University of Texas System, Austin, TX (US)

(21) Appl. No.: 17/907,031

(22) PCT Filed: Mar. 23, 2021
(86) PCT No.: PCT/US2021/023731

§ 371 Date: Sep. 22, 2022

Related U.S. Application Data
(60) Provisional application No. 62/993,424, filed on Mar. 23, 2020.

(51) Int. Cl. A61K 39/215; A61K 9/127; C12N 15/113; A61P 31/14

(52) U.S. Cl. CPC A61K 39/215; A61K 9/1271; C12N 15/1137; C12N 15/1131; A61P 31/14; A61K 2039/54

(57) ABSTRACT

The current disclosure provides compositions and methods for treatment and prevention of a coronavirus infection. Certain aspects are directed to lipid vesicles comprising SARS-CoV-2 spike protein or a portion or variant thereof. Further aspects include methods for treatment or prevention of a coronavirus infection comprising providing a lipid vesicle comprising a therapeutic protein, such as SARS-CoV-2 spike protein or ACE2. In some embodiments, the disclosed lipid vesicles are useful for targeted delivery of anti-viral therapeutic agents.

This application is claims benefit of priority of U.S. Provisional Application No. 62/993,424, filed Mar. 23, 2020, which is hereby incorporated by reference in its entirety.

### BACKGROUND OF THE INVENTION

### I. Field of the Invention

Aspects of the invention relate to the fields of immunology, virology, and molecular biology.

### II. Background

A disease known as COVID-19 is caused by a novel coronavirus, SARS-CoV-2 (also 2019-nCoV) and is associated with fever, severe respiratory illness, and pneumonia. This viral pathogen is a new member of the betacaronavirus genus and shows high degree of homology with SARS coronavirus (SARS-CoV). SARS-CoV-2 is a RNA virus with a positive sense single stranded RNA as its genome (+ssRNA) and is about 30,000 bases in length. Each SARS-CoV-2 is 50-200 nm in diameter. SARS-CoV-2 has four structural proteins that make up the entire virus structure/particle. The Nucleocapsid protein (N protein) interacts provides scaffold for the RNA genome. The Spike (S), membrane (M) and envelope (E) glycoproteins provide the structural basis for the viral envelope. The S glycoprotein participates in binding to the membrane of the host human cells, such as respiratory cells in the lung and facilitate attachment of the virus and cellular entry via receptor mediated endocytosis.

The receptor on the host human cells that binds to the S glycoprotein on the surface of the SARS-CoV-2 virus is known as angiotensin converting enzyme-2 (ACE2). This high affinity binding leads to viral entry into the cell. The S1 domain of the S glycoprotein binds to the peptidase domain of ACE2 on the cell surface and the S2 domain of the S glycoprotein allows for membrane binding. ACE2 is a type I transmembrane protein receptor expressed in lung, GI tract, kidneys, and heart. ACE2 cleaves angiotensin to generate and active a peptide that controls vasoconstriction and regulates blood pressure.

There is a pressing need for methods and compositions for treatment and prevention of SARS-CoV-2 infection.

### SUMMARY OF THE INVENTION

Aspects of the disclosure are directed to lipid vesicles, such as exosomes, comprising one or more therapeutic proteins for treatment or prevention of a coronavirus (e.g., SARS-CoV2 coronavirus) infection. Lipid vesicles of the disclosure comprise therapeutic proteins expressed on their surface. Therapeutic proteins may comprise a transmembrane domain and an extracellular domain. In some embodiments, a lipid vesicle comprises a coronavirus spike protein or a portion thereof (e.g., S1 domain, S2 domain). In some embodiments, a lipid vesicle comprises a protein which facilitates coronavirus entry into a cell, for example angiotensin converting enzyme 2 (ACE2) or a portion thereof (e.g., PD domain, CLD domain).

Embodiments of the disclosure include lipid vesicles; liposomes; exosomes; anti-viral therapeutics; cells configured to produce lipid vesicles; cells configured to produce liposomes; cells configured to produce exosomes; vaccine compositions; vaccine compositions comprising lipid vesicles; vaccine compositions comprising liposomes; vaccine compositions comprising exosomes; vaccine compositions comprising cells configured to produce lipid vesicles; vaccine compositions comprising cells configured to produce liposomes; vaccine compositions comprising cells configured to produce exosomes; therapeutic proteins; coronavirus spike proteins; angiotensin converting enzyme 2 (ACE2) proteins; lipid vesicles comprising therapeutic proteins; liposomes comprising therapeutic proteins; exosomes comprising therapeutic proteins; cells configured to produce lipid vesicles comprising therapeutic proteins; cells configured to produce liposomes comprising therapeutic proteins; cells configured to produce exosomes comprising therapeutic proteins; vaccine compositions comprising lipid vesicles comprising therapeutic proteins; vaccine compositions comprising liposomes comprising therapeutic proteins; vaccine compositions comprising exosomes comprising therapeutic proteins; vaccine compositions comprising cells configured to produce lipid vesicles comprising therapeutic proteins; vaccine compositions comprising cells configured to produce liposomes comprising therapeutic proteins; vaccine compositions comprising cells configured to produce exosomes comprising therapeutic proteins; lipid vesicles comprising coronavirus spike proteins; liposomes comprising coronavirus spike proteins; exosomes comprising coronavirus spike proteins; cells configured to produce lipid vesicles comprising coronavirus spike proteins; cells configured to produce liposomes comprising coronavirus spike proteins; cells configured to produce exosomes comprising coronavirus spike proteins; vaccine compositions comprising lipid vesicles comprising coronavirus spike proteins; vaccine compositions comprising liposomes comprising coronavirus spike proteins; vaccine compositions comprising exosomes comprising coronavirus spike proteins; vaccine compositions comprising cells configured to produce lipid vesicles comprising coronavirus spike proteins; vaccine compositions comprising cells configured to produce liposomes comprising coronavirus spike proteins; vaccine compositions comprising cells configured to produce exosomes comprising coronavirus spike proteins; lipid vesicles comprising ACE2 proteins; liposomes comprising ACE2 proteins; exosomes comprising ACE2 proteins; cells configured to produce lipid vesicles comprising ACE2 proteins; cells configured to produce liposomes comprising ACE2 proteins; cells configured to produce exosomes comprising ACE2 proteins; vaccine compositions comprising lipid vesicles comprising ACE2 proteins; vaccine compositions comprising liposomes comprising ACE2 proteins; vaccine compositions comprising exosomes comprising ACE2 proteins; vaccine compositions comprising cells configured to produce lipid vesicles comprising ACE2 proteins; vaccine compositions comprising cells configured to produce liposomes comprising ACE2 proteins; vaccine compositions comprising cells configured to produce exosomes comprising ACE2 proteins; methods for treatment of a coronavirus infection; methods for treatment of a SARS-CoV-2 infection; methods for prevention of a coronavirus infection; methods for prevention of a SARS-CoV-2 infection; methods for reducing severity of a coronavirus infection; methods for reducing severity of a SARS-CoV-2 infection; methods for delaying the onset of a coronavirus infection; methods for delaying the onset of a SARS-CoV-2 infection; methods for inhibiting entry of a coronavirus into a cell; methods for inhibiting entry of a SARS-CoV-2 virus into a cell; methods for inhibiting an interaction between a coronavirus spike protein and host cell membrane; methods for inhibiting an interaction between a SARS-CoV-2 spike protein and a host cell membrane; pharmaceutical compositions; and kits. Because the SARS-CoV-2 virus causes COVID-19, any embodiment discussed in the context of SARS-CoV-2 can be implemented with respect to COVID-19.

Methods of the disclosure can include 1, 2, 3, 4, 5, 6, or more of the following steps: administering a lipid vesicle to a subject, administering a liposome to a subject, administering a exosome to a subject, administering an anti-viral therapeutic to a subject, diagnosing a subject as having a coronavirus infection, diagnosing a subject as having a SARS-CoV-2 infection, diagnosing a subject as having symptoms of a coronavirus infection, diagnosing a subject as having symptoms of a SARS-CoV-2 infection, diagnosing a subject as being at risk of having a coronavirus infection, diagnosing a subject as being at risk of having a SARS-CoV-2 infection, obtaining a sample from a subject, detecting a coronavirus in a sample, detecting a SARS-CoV-2 virus in a sample, and providing two or more types of anti-viral therapy to a subject. Certain embodiments of the disclosure may exclude one or more of the preceding elements and/or steps.

Compositions of the disclosure can include at least 1, 2, 3, 4, 5, or more of the following components: lipid vesicles; liposomes; exosomes; anti-viral therapeutics; cells configured to produce lipid vesicles; cells configured to produce liposomes; cells configured to produce exosomes; vaccine compositions; vaccine compositions comprising lipid vesicles; vaccine compositions comprising liposomes; vaccine compositions comprising exosomes; vaccine compositions comprising cells configured to produce lipid vesicles; vaccine compositions comprising cells configured to produce liposomes; vaccine compositions comprising cells configured to produce exosomes; therapeutic proteins; coronavirus spike proteins; angiotensin converting enzyme 2 (ACE2) proteins; lipid vesicles comprising therapeutic proteins; liposomes comprising therapeutic proteins; exosomes comprising therapeutic proteins; cells configured to produce lipid vesicles comprising therapeutic proteins; cells configured to produce liposomes comprising therapeutic proteins; cells configured to produce exosomes comprising therapeutic proteins; pharmaceutically acceptable carriers; and excipients. One or more of these components may be specifically excluded from certain embodiments.

Disclosed herein, in some embodiments, is a lipid vesicle comprising a SARS-CoV-2 spike protein, or portion or variant thereof, comprising a transmembrane domain and an external domain, wherein the external domain is on an exterior surface of the lipid vesicle. In some embodiments, the external domain comprises an S1 domain and/or an S2 domain. In some embodiments, the lipid vesicle is a liposome. In some embodiments, the lipid vesicle is an exosome.

Also disclosed herein is a cell configured to produce a lipid vesicle of the present disclosure. In some embodiments, the cell is a mesenchymal stem cell. Further disclosed is a method for preventing or reducing a SARS-CoV-2 infection, the method comprising providing to a subject an effective amount of a composition comprising a lipid vesicle of the present disclosure.

Also disclosed herein, in some embodiments, is a method for treating or preventing a coronavirus infection, the method comprising providing to a subject an effective amount of a composition comprising a lipid vesicle comprising a therapeutic protein comprising a transmembrane domain and an external domain, wherein the external domain is on an exterior surface of the lipid vesicle. In some embodiments, the coronavirus is a betacoronavirus. In some embodiments, the coronavirus is SARS-CoV-2. In some embodiments, the therapeutic protein is a coronavirus spike protein or a portion or variant thereof. In some embodiments, the therapeutic protein is an angiotensin converting enzyme 2 (ACE2) protein or portion or variant thereof. In some embodiments, the external domain comprises a PD domain and/or a CLD domain. In some embodiments, the method comprises generating antibodies against the therapeutic protein in the subject. In some embodiments, the subject is infected with the coronavirus.

In some embodiments, the subject is at risk for the SARS-CoV-2 infection. In some embodiments, the composition further comprises a vaccine adjuvant, which may be aluminum of a lipid-based adjuvant. In some embodiments, the composition is provided via intranasal, intraperitoneal, or intramuscular administration. In some embodiments, the subject is infected with the SARS-CoV-2.

In some embodiments, the lipid vesicle further comprises an anti-viral therapeutic. In some embodiments, the anti-viral therapeutic is a nucleic acid, which may be a small interfering RNA (siRNA), a small hairpin RNA (shRNA), or an antisense oligonucleotide. In some embodiments, the anti-viral nucleic acid is configured to target and reduce expression of a host protein, wherein the host protein is ACE2, TMPRSS2, 3CLpro, ALpro, or AT2. In some embodiments, the host protein is ACE2. In some embodiments, the anti-viral nucleic acid is configured to target and reduce expression of a viral protein, wherein the viral protein is a spike protein, an envelope protein, a membrane glycoprotein, a nucleocapsid protein, an RNA-dependent RNA polymerase, a replicase, or a helicase. In some embodiments, the anti-viral therapeutic is an anti-viral compound. In some embodiments, the anti-viral compound is an RNA polymerase inhibitor, a replicase inhibitor, or a helicase inhibitor. In some embodiments, the lipid vesicle does not comprise an anti-viral therapeutic.

In some embodiments, the method comprises providing a cell configured to produce the lipid vesicle. In some embodiments, the cell is a mesenchymal stem cell. In some embodiments, the cell is from a mammalian cell line. In some embodiments, the cell is a 293T cell. In some embodiments, the cell is a 293F cell.

Also disclosed herein, in some embodiments, is a method for treating or preventing a SARS-CoV-2 infection, the method comprising providing to a subject an effective amount of a composition comprising a lipid vesicle comprising ACE2 or a portion or variant thereof. In some embodiments, the lipid vesicle comprises a portion of ACE2. In some embodiments, the portion of ACE2 comprises a PD domain. In some embodiments, the portion of ACE2 comprises a CLD domain. In some embodiments, the external domain comprises a PD domain and a CLD domain. In some embodiments, the lipid vesicle comprises ACE2.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

Use of the one or more sequences or compositions may be employed based on any of the methods described herein. Other embodiments are discussed throughout this application. Any embodiment discussed with respect to one aspect of the disclosure applies to other aspects of the disclosure as well and vice versa. For example, any step in a method described herein can apply to any other method. Moreover, any method described herein may have an exclusion of any step or combination of steps. The embodiments in the Examples section are understood to be embodiments that are applicable to all aspects of the technology described herein.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows a schematic of a SARS-CoV-2 coronavirus, including certain viral membrane proteins.

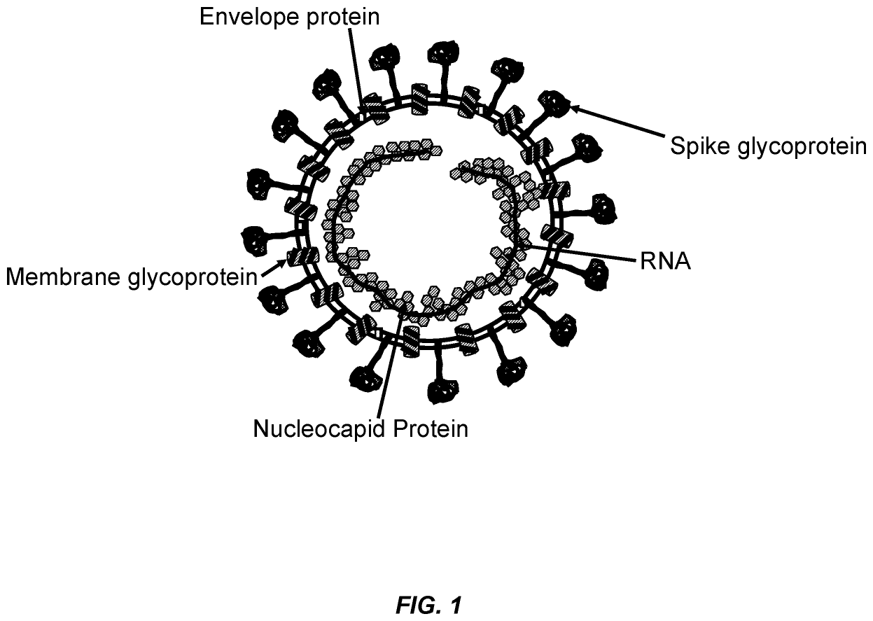

FIG. 2 shows a schematic of example lipid vesicles comprising coronavirus spike protein (S protein) on their surface.

FIG. 3 shows a schematic of example lipid vesicles comprising angiotensin converting enzyme 2 (ACE2) on their surface.

FIG. 4 shows a schematic representation of two versions of an Exo^{2019 nCov S protein}. Top: representation of the SARS-CoV-2 Spike (S) protein with main domains (including RBD) indicated. Bottom left: Representation of Exo-SC2S exosomes expressing full length S protein. Bottom right: Representation of Exo-SC2S-RBD exosomes expressing the RBD of SARS-CoV-2 on a VSV-G-presenting protein.

FIGS. 5A-5B show production of Exo^{2019 nCov S protein}. FIG. 5A. Nanosight data showing the concentration versus size (nm) of exosomes, Exo-SC2S exosomes expressing full length S protein, and Exo-SC2S-RBD exosomes expressing the RBD of SARS-CoV-2 on a VSV-G-presenting protein. Inset are representative electron microscopy images of exosomes and Exo-SC2S exosomes expressing full length S protein. FIG. 5B. Exosome production for exosomes, Exo-SC2S exosomes expressing full length S protein, and Exo-SC2S-RBD exosomes expressing the RBD of SARS-CoV-2 on a VSV-G-presenting protein shown as nanosight measurement of exosome concentrations normalized to the number of cells producing them (left; exosomes/cell) and as protein measurements normalized to 1 million cells (right; exosome protein per 10⁶ cells).

FIGS. 6A-6B show validation of S protein expression by Exo-SC2S exosomes. FIG. 6A. Western blot analysis for detection of S-Protein in exosomes and Exo-SC2S exosomes expressing SC2S; CD81: loading control. FIG. 6B. Flow cytometry analysis of exosomes and Exo-SC2S exosomes expressing SC2S for S-protein and for the exosome markers CD9, CD63, CD81.

FIG. 7 illustrates a schematic of the ELISA assay used to quantify spike protein levels in engineered Exo-SC2S exosomes expressing full length S protein and Exo-SC2S-RBD exosomes expressing the RBD of SARS-CoV-2 on a VSV-G-presenting protein.

FIGS. 8A-8C show validation of substrate binding by Exo-SC2S exosomes. FIGS. 8A-8B. Results from ELISA assays showing dose response binding to increasing RBD substrate (FIG. 8A) or Exo-SC2S substrate concentrations. FIG. 8C. Concentration of S-protein (μg) expressed by increasing concentrations of Exo-SC2S exosomes (μg).

FIGS. 9A-9C show validation of substrate binding by Exo-SC2S-RBD exosomes. FIGS. 9A-9B. Results from ELISA assays showing dose response binding to increasing RBD substrate (FIG. 9A) or Exo-SC2S-RBD substrate concentrations. FIG. 9C. Concentration of S-protein (μg) expressed by increasing concentrations of Exo-SC2S-RBD exosomes (μg).

FIGS. 10A-10B show a schematic of the experimental timeline for administration of Exo^{2019 nCov S protein} to mice (FIG. 10A) and the percent body weight change in mice administered control (PBS), exosomes, Exo-SC2S, Exo-SC2S-RBD, or 1 μg or 10 μg SC2S (FIG. 10B).

FIGS. 11A-11B show antibody generation against SARS-CoV-2 spike protein RBD in mice following intramuscular vaccination with Exo^{2019 nCov S protein}. ELISA-based detection of IgM (FIG. 11A) and IgG (FIG. 11B) antibody production in the blood of mice following intramuscular vaccination. Exo-SC2S and Exo-SC2S-RBD exosomes result in antibody production. Exo-SC2S exosome-induced antibody production was significant compared to control exosome-induced antibody production.

FIGS. 12A-12B show that antibodies generated in Exo-SC2S-vaccinated mice are neutralizing. FIG. 12A. Schematic of assay utilizing pseudoviruses to determine whether antibodies produced as a result of Exo-SC2S vaccination are neutralizing. FIG. 12B. Results from the neutralizing assay indicates antibodies in the blood of mice following Exo-SC2S intramuscular vaccination show neutralizing activity.

FIGS. 13A-13B show generation of Exo-SC2S and Exo-SC2S-RBD exosomes by 293F cells. For GMP production, 293F cells are used. The 293F cells were engineered to produce the Exo-SC2S Exo^{2019 nCov S protein} and Exo-SC2S-RBD Exo^{2019 nCov S protein}. Western blot analysis showing detection of S-protein (Exo-SC2S Exo^{2019 nCov S protein}, FIG. 13A) and RBD (Exo-SC2S-RBD Exo^{2019 nCov S protein} FIG. 13B). CD81: loading control.

FIG. 14 shows validation of Exo-ACE2 exosome generation by 293T cells by western blot analysis of the 293T cells. β-actin: loading control.

### DETAILED DESCRIPTION OF THE INVENTION

The present disclosure provides methods and compositions for treatment and prevention of a coronavirus infection, including SARS-CoV-2. Particular aspects are directed to lipid vesicles, for example exosomes, which express on their surface one or more therapeutic proteins capable of preventing infection or transmission of a coronavirus. Examples of therapeutic proteins useful in treatment of coronavirus infection include coronavirus spike (S) protein and angiotensin converting enzyme 2 (ACE2). Lipid vesicles of the disclosure are useful as, for example, a vaccine for preventing or reduction of a coronavirus infection or as a therapeutic for treatment of an ongoing coronavirus infection. For example, a lipid vesicle expressing a coronavirus spike protein may be used as a vaccine and/or as a decoy for binding to ACE2-expressing cells, thereby preventing entry of a coronavirus (e.g., SARS-CoV-2). In other examples, a lipid vesicle expressing an ACE2 protein may be used as a decoy receptor for binding to and preventing entry of a coronavirus. In some examples, disclosed are methods of delivering an anti-viral therapeutic to a cell using a lipid vesicle expressing a coronavirus spike protein (e.g., SARS-CoV-2 spike protein).

### I. Viruses

Aspects of the present disclosure relate to treatment or prevention of a virus. In some embodiments, disclosed are methods for treatment or prevention of a viral infection. In some embodiments, disclosed are compositions comprising one or more anti-viral agents.

In particular embodiments, the virus is from the family Coronaviridae. Coronaviridae is a family of enveloped, positive-sense, single-stranded RNA viruses. Coronavirus is the common name for Coronaviridae and Orthocoronavirinae (also referred to as Coronavirinae). The family Coronaviridae is organized in 2 sub-families, 5 genera, 23 sub-genera and approximately 40 species. They are enveloped viruses having a positive-sense single-stranded RNA genome and a nucleocapsid having helical symmetry.

Several coronaviruses utilize animals as their primary hosts and have also evolved to infect humans. There are four main sub-groupings of coronaviruses, known as alpha, beta, gamma, and delta, and seven coronaviruses that can infect people. The four most common coronaviruses utilize humans as their natural host and include: 229E (alpha coronavirus); NL63 (alpha coronavirus); OC43 (beta coronavirus); HKU1 (beta coronavirus). Three other human coronaviruses are: MERS-CoV (the beta coronavirus that causes MERS); SARS-CoV (the beta coronavirus that causes SARS); and SARS-CoV-2 (the novel coronavirus that causes coronavirus disease 2019, or COVID-19).

Coronaviruses have characteristic club-shaped spikes that project from their surface, which in electron micrographs create an image reminiscent of the solar corona, from which their name derives. The average diameter of the virus particles is around 120 nm (0.12 m). The diameter of the envelope is ˜80 nm (0.08 m) and the spikes are ˜20 nm (0.02 m) long. Beneath the spiked exterior of the virus is a round core shrouded in a viral envelope. The core contains genetic material that the virus can inject into cells to infect them.

The viral envelope consists of a lipid bilayer where the membrane (M), envelope (E), and spike (S) structural proteins are anchored. Inside the envelope, there is the nucleocapsid of helical symmetry which is formed from multiple copies of the nucleocapsid (N) protein, which are bound to the positive-sense single-stranded RNA genome in a continuous beads-on-a-string type conformation. The genome size of coronaviruses ranges from approximately 26 to 32 kilobases. The genome organization for a coronavirus is 5′-leader-UTR-replicase/transcriptase-spike (S)-envelope (E)-membrane (M)-nucleocapsid (N)-3′UTR-poly (A) tail. The open reading frames 1a and 1b, which occupy the first two-thirds of the genome, encode the replicase/transcriptase polyprotein. The replicase/transcriptase polyprotein self cleaves to form nonstructural proteins. The later reading frames encode the four major structural proteins: spike, envelope, membrane, and nucleocapsid. Interspersed between these reading frames are the reading frames for the accessory proteins. The number of accessory proteins and their function is unique depending on the specific coronavirus.

The lipid bilayer envelope, membrane proteins, and nucleocapsid protect the virus when it is outside the host cell. The spike proteins extend from within the core to the viral surface and allow the virus to recognize and bind specific cells in the body. When the spike engages a receptor on a host cell, a cascade is triggered, resulting in the merger of the virus with the cell which allows the virus to release its genetic material and overtake the cell's processes to produce new viruses.

Infection begins when the viral spike (S) glycoprotein attaches to its complementary host cell receptor. After attachment, a protease of the host cell cleaves and activates the receptor-attached spike protein. Depending on the host cell protease available, cleavage and activation allows the virus to enter the host cell by endocytosis or direct fusion of the viral envelop with the host membrane. On entry into the host cell, the virus particle is uncoated, and its genome enters the cell cytoplasm. The coronavirus RNA genome has a 5′ methylated cap and a 3′ polyadenylated tail, which allows the RNA to attach to the host cell's ribosome for translation. The host ribosome translates the initial overlapping open reading frame of the virus genome and forms a long polyprotein. The polyprotein has its own proteases which cleave the polyprotein into multiple nonstructural proteins.

Viral entry is followed by replication of the virus. A number of the nonstructural proteins coalesce to form a multi-protein replicase-transcriptase complex (RTC). The main replicase-transcriptase protein is the RNA-dependent RNA polymerase (RdRp). It is directly involved in the replication and transcription of RNA from an RNA strand. The other nonstructural proteins in the complex assist in the replication and transcription process. The exoribonuclease nonstructural protein, for instance, provides extra fidelity to replication by providing a proofreading function which the RNA-dependent RNA polymerase lacks. One of the main functions of the complex is to replicate the viral genome. RdRp directly mediates the synthesis of negative-sense genomic RNA from the positive-sense genomic RNA. This is followed by the replication of positive-sense genomic RNA from the negative-sense genomic RNA. The other important function of the complex is to transcribe the viral genome. RdRp directly mediates the synthesis of negative-sense subgenomic RNA molecules from the positive-sense genomic RNA. This is followed by the transcription of these negative-sense subgenomic RNA molecules to their corresponding positive-sense mRNAs.

The replicated positive-sense genomic RNA becomes the genome of the progeny viruses. The mRNAs are gene transcripts of the last third of the virus genome after the initial overlapping reading frame. These mRNAs are translated by the host's ribosomes into the structural proteins and a number of accessory proteins. RNA translation occurs inside the endoplasmic reticulum. The viral structural proteins S, E, and M move along the secretory pathway into the Golgi intermediate compartment. There, the M proteins direct most protein-protein interactions required for assembly of viruses following its binding to the nucleocapsid. Progeny viruses are then released from the host cell by exocytosis through secretory vesicles.

The interaction of the coronavirus spike protein with its complement host cell receptor is central in determining the tissue tropism, infectivity, and species range of the virus. Coronaviruses mainly target epithelial cell receptors. They can be transmitted by aerosol, fomite, or fecal-oral routes, for example. Human coronaviruses infect the epithelial cells of the respiratory tract, while animal coronaviruses generally infect the epithelial cells of the digestive tract. For example, coronaviruses such as SARS-CoV-2 can infect, via an aerosol route, human epithelial cells of the lungs by binding of the spike protein receptor binding domain (RBD) to an angiotensin-converting enzyme 2 (ACE2) receptor on the cell surface.

The WHO has reported that the two groups most at risk of experiencing severe illness due to a coronavirus infection and/or post-coronavirus infection syndrome are adults aged 65 years or older and people who have other underlying health conditions including chronic lung disease, serious heart conditions, severe obesity, a compromised immune system, or diabetes. In humans, coronaviruses typically cause a respiratory infection with mild to severe flu-like symptoms, but the exact symptoms vary depending on the type of coronavirus. The four common human coronaviruses can cause people to develop a runny nose, headache, cough, sore throat and fever. In a subset of subjects, including those with cardiopulmonary disease or a weakened immune system, the viral infection can progress to a more severe lower-respiratory infection such as pneumonia or bronchitis. In comparison, severe MERS and SARS infections often progress to pneumonia. Other symptoms of MERS include fever, coughing, and shortness of breath, while SARS can cause fever, chills and body aches.

Coronaviruses cause a variety of symptoms, triggering fever, cough, and shortness of breath in most patients. Rarer symptoms include dizziness, tiredness, aches, chills, sore throat, loss of smell, loss of taste, headache, nausea, vomiting, and diarrhea. Emergency signs or symptoms can include trouble breathing, persistent chest pain or pressure, new confusion, and/or blue lips or face. Complications of coronavirus infections can include pneumonia, organ failure, respiratory failure, blood clots, heart conditions such as cardiomyopathies, acute kidney injury, and/or further viral and bacterial infections.

The present disclosure encompasses treatment or prevention of infection of any virus in the Coronaviridae family. In certain embodiments, the disclosure encompasses treatment or prevention of infection of any virus in the subfamily Coronavirinae and including the four genera, Alpha-, Beta-, Gamma-, and Deltacoronavirus. In specific embodiments, the disclosure encompasses treatment or prevention of infection of any virus in the genus of Betacoronavirus, including the subgenus Sarbecovirus and the species severe acute respiratory syndrome-related coronavirus; the subgenus Embecovirus and the species human coronavirus HKU1; and the species Betacoronavirus 1. In specific embodiments, the disclosure encompasses treatment or prevention of infection of any virus in the species of severe acute respiratory syndrome-related coronavirus, including severe acute respiratory syndrome coronavirus 2 (SARS-CoV-2, the virus that causes COVID-19). The disclosure encompasses treatment or prevention of infection any isolate, strain, type (including Type A, Type B and Type C; Forster et al., 2020, PNAS, available on the World Wide Web at doi.org/10.1073/pnas.2004999117), cluster, or sub-cluster of the severe acute respiratory syndrome-related coronavirus, including at least SARS-CoV-2. In specific embodiments, the virus has a genome length between 29000 to 30000, between 29100 and 29900, between 29200 and 29900, between 29300 and 29900, between 29400 and 29900, between 29500 and 29900, between 29600 and 29900, between 29700 and 29900, between 29800 and 29900, or between 29780 and 29900 base pairs in length.

### II. Anti-Viral Treatment

Compositions (e.g., lipid vesicles, liposomes, or exosomes; cells comprising lipid vesicles, liposomes, or exosomes; vaccine compositions comprising lipid vesicles, liposomes, or exosomes; vaccine compositions comprising cells comprising lipid vesicles, liposomes, or exosomes) or methods described herein may be administered to any subject having a condition in which targeting host and/or viral proteins may have therapeutic benefit. Conditions in which targeting host and/or viral proteins may have a therapeutic benefit include, for example, a condition associated with binding of viral particles to cells and entry of viral particles into cells. Such conditions include, for example, coronavirus infection.

As used herein, “coronavirus infection” refers to an infection caused by any Coronaviridae family member. For example, coronavirus infections can include but are not limited to SARS-CoV-2 infections. Thus, aspects of the present disclosure are directed to methods comprising treatment of a subject suffering from, suspected of having, or at risk for developing a coronavirus infection. In some embodiments, the coronavirus infection is a SARS-CoV-2 infection.

In specific embodiments, the methods and compositions comprise treating, preventing, delaying onset of, and/or reducing severity of a coronavirus infection in an subject in need thereof by administering an effective amount of a lipid vesicle comprising a SARS-CoV-2 spike protein, or portion or variant thereof, comprising a transmembrane domain and an external domain, wherein the external domain is on an exterior surface of the lipid vesicle. In specific embodiments, the methods and compositions comprise treating, preventing, delaying onset of, and/or reducing severity of a coronavirus infection in an subject in need thereof by administering an effective amount of cells configured to produce a lipid vesicle comprising a SARS-CoV-2 spike protein, or portion or variant thereof, comprising a transmembrane domain and an external domain, wherein the external domain is on an exterior surface of the lipid vesicle. In specific embodiments, the methods and compositions comprise treating, preventing, delaying onset of, and/or reducing severity of a coronavirus infection in an subject in need thereof by administering an effective amount of a lipid vesicle comprising a therapeutic protein comprising a transmembrane domain and an external domain, wherein the external domain is on an exterior surface of the lipid vesicle. In specific embodiments, the methods and compositions comprise treating, preventing, delaying onset of, and/or reducing severity of a coronavirus infection in an subject in need thereof by administering an effective amount of cells configured to produce a lipid vesicle comprising a therapeutic protein comprising a transmembrane domain and an external domain, wherein the external domain is on an exterior surface of the lipid vesicle.

In specific embodiments, the effective amount is effective to treat, prevent, delay onset of, and/or reduce severity of a coronavirus infection in the subject. In specific embodiments, the methods and compositions further comprise increasing the survival rate of a subject infected with a coronavirus. In specific embodiments, the methods and compositions further comprise reducing the recovery time of a subject infected with a coronavirus. In specific embodiments, the methods and compositions further comprise treating, preventing, delaying onset of, and/or reducing severity of the symptoms of a coronavirus infection in a subject. In specific embodiments, the methods and compositions further comprise treating, preventing, delaying onset of, and/or reducing severity of cellular, tissue, organ, or system damage caused by a coronavirus infection in a subject.

A subject in need thereof may be a subject having one or more symptoms of infection by a virus of the Coronaviridae family, such as SARS-CoV-2. Common initial signs and symptoms of SARS-CoV-2 may include fever, cough, shortness of breath or difficulty breathing, tiredness, aches, chills, sore throat, loss of smell, loss of taste headache, diarrhea, or vomiting. As the viral infection progresses, the individual may develop pneumonia or acute respiratory distress syndrome (ARDS).

In specific embodiments, a subject may be diagnosed with a viral infection based on the onset of symptoms of the viral infection; and/or based on a positive biological test for a current viral infection. In specific embodiments, the biological test for a current viral infection is an assay for the virus. In specific embodiments, a subject may be considered recovered from a viral infection based on the amount of time which has passed since the onset of symptoms of the viral infection, the amount of time which has passed without a fever in the absence of use of fever-reducing medication, and the improvement of other symptoms of the viral infection; and/or two consecutive negative biological tests for a current viral infection taken at least a certain time period apart. In specific embodiments, a subject may be considered recovered from a coronavirus infection if at least 10 days have passed since coronavirus infection symptoms first appeared, at least 24 hours have passed with no fever without the use of fever-reducing medications, and other symptoms of coronavirus infection are improving; and/or two biological test for a current coronavirus infection taken at least 24 hours apart are both negative. In specific embodiments, a subject may confirm a previous viral infection based on a biological test for a past viral infection. In specific embodiments, the biological test for a past viral infection is an assay for viral antibodies. In specific embodiments, the biological test for a past coronavirus infection is an assay for Coronaviridae family viral antibodies. In specific embodiments, a subject considered recovered from a viral infection may be diagnosed with a post-viral infection syndrome based on persistent symptoms of the viral infection and/or chronic effects of cellular, tissue, organ, or system damage caused by the viral infection. In specific embodiments, persistent symptoms of a coronavirus infection and/or chronic effects of cellular, tissue, organ, or system damage caused a coronavirus infection include persistent fever, cough, shortness of breath, difficulty breathing, tiredness, aches, chills, sore throat, loss of smell, loss of taste, headache, diarrhea, vomiting, pneumonia, acute respiratory distress syndrome (ARDS), dizziness, mood disorders, cognitive impairment, muscle weakness, nerve damage, joint pain, chest pain, palpitations, rash, hair loss, worsened quality of life, lung damage, heart damage, heart swelling, kidney damage, or liver damage. In specific embodiments, a subject in need thereof may be a subject having one or more persistent symptoms of a coronavirus infection, such as SARS-CoV-2, and/or chronic effects of chronic effects of cellular, tissue, organ, or system damage caused by a coronavirus infection, such as SARS-CoV-2. Common persistent symptoms of a coronavirus infection, such as SARS-CoV, SARS-CoV-2, or MERS-CoV, and/or chronic effects of chronic effects of cellular, tissue, organ, or system damage caused by a coronavirus infection, such as SARS-CoV-2, may include persistent fever, cough, shortness of breath, difficulty breathing, tiredness, aches, chills, sore throat, loss of smell, loss of taste, headache, diarrhea, vomiting, pneumonia, acute respiratory distress syndrome (ARDS), dizziness, mood disorders, cognitive impairment, muscle weakness, nerve damage, joint pain, chest pain, palpitations, rash, hair loss, worsened quality of life, lung damage, heart damage, heart swelling, kidney damage, or liver damage.

In some embodiments of the methods disclosed herein, the subject is at high risk for having coronavirus infection. In some embodiments, the subject does not have a coronavirus infection or has tested negative for a coronavirus infection. In some embodiments, the subject was diagnosed as having a coronavirus infection. In some embodiments, the subject is diagnosed as having symptoms of the coronavirus infection. In some embodiments, the subject is diagnosed as being at risk of having the coronavirus infection. In some embodiments, the subject has severe acute respiratory syndrome (SARS) or a respiratory infection. In some embodiments, the subject has COVID-19.

The term “treatment” or “treating” means any treatment of a disease in a mammal, including: (i) preventing the disease, that is, causing the clinical symptoms of the disease not to develop by administration of a protective composition prior to the induction of the disease; (ii) suppressing the disease, that is, causing the clinical symptoms of the disease not to develop by administration of a protective composition after the inductive event but prior to the clinical appearance or reappearance of the disease; (iii) inhibiting the disease, that is, arresting the development of clinical symptoms by administration of a protective composition after their initial appearance; and/or (iv) relieving the disease, that is, causing the regression of clinical symptoms by administration of a protective composition after their initial appearance.

The therapy provided herein may comprise administration of a composition comprising a therapeutic agent (e.g., lipid vesicles, liposomes, or exosomes; cells comprising lipid vesicles, liposomes, or exosomes; vaccine compositions comprising lipid vesicles, liposomes, or exosomes; vaccine compositions comprising cells comprising lipid vesicles, liposomes, or exosomes). In some embodiments, therapy provided herein comprises administration of lipid vesicles, liposomes, or exosomes and a pharmaceutically acceptable excipient. In some embodiments, therapy provided herein comprises administration of cells comprising lipid vesicles, liposomes, or exosomes and a pharmaceutically acceptable excipient. In some embodiments, therapy provided herein comprises administration of vaccine compositions comprising lipid vesicles, liposomes, or exosomes and a pharmaceutically acceptable excipient. In some embodiments, therapy provided herein comprises administration of vaccine compositions comprising cells comprising lipid vesicles, liposomes, or exosomes and a pharmaceutically acceptable excipient.

In some embodiments, the disclosed methods comprise treating a subject suffering from a coronavirus infection, e.g., a SARS-CoV-2 infection, with lipid vesicles, liposomes, or exosomes; cells comprising lipid vesicles, liposomes, or exosomes; vaccine compositions comprising lipid vesicles, liposomes, or exosomes; or vaccine compositions comprising cells comprising lipid vesicles, liposomes, or exosomes. The lipid vesicles, liposomes, or exosomes may be engineered to overexpress host or viral proteins. In some embodiments, the lipid vesicles, liposomes, or exosomes are engineered to overexpress viral spike protein. In some embodiments, the lipid vesicles, liposomes, or exosomes are engineered to overexpress ACE2 protein. As disclosed herein, when the lipid vesicles, liposomes, or exosomes are engineered to overexpress viral spike protein, administration of the lipid vesicles, liposomes, or exosomes; cells comprising lipid vesicles, liposomes, or exosomes; vaccine compositions comprising lipid vesicles, liposomes, or exosomes; or vaccine compositions comprising cells comprising lipid vesicles, liposomes, or exosomes, the lipid vesicles, liposomes, or exosomes can serve as decoy for ACE2, thereby preventing S protein on SARS CoV-2 virus binding to ACE2 and suppressing internalization of the virus and its subsequent multiplication. Accordingly, in some embodiments, disclosed is a method for treating or preventing a coronavirus infection, the method comprising providing to a subject an effective amount of a composition comprising a lipid vesicle comprising a therapeutic protein comprising a transmembrane domain and an external domain, wherein the external domain is on an exterior surface of the lipid vesicle. In some embodiments, the coronavirus is a betacoronavirus. In some embodiments, the coronavirus is SARS-CoV-2. In some embodiments, the therapeutic protein is a coronavirus spike protein or portion or variant thereof, and the external domain comprises an S1 domain, an S2 domain, or an S1 domain and an S2 domain. In other embodiments, the therapeutic protein is an angiotensin converting enzyme 2 (ACE2) protein or portion or variant thereof, and the external domain comprises a PD domain, a CLD domain, or a PD domain and a CLD domain.

Also disclosed herein, when the lipid vesicles, liposomes, or exosomes are engineered to overexpress viral spike protein, administration of the lipid vesicles, liposomes, or exosomes; cells comprising lipid vesicles, liposomes, or exosomes; vaccine compositions comprising lipid vesicles, liposomes, or exosomes; or vaccine compositions comprising cells comprising lipid vesicles, liposomes, or exosomes, the lipid vesicles, liposomes, or exosomes allows for binding of ACE2 on cell expressing the viral spike proteins, which allows internalization of the exosomes, therefore allowing for delivery of drug payload that can interfere with viral replication and assembly and/or neutralize host proteins that aid in the virus propagation. Accordingly, in some embodiments, disclosed is a method for treating or preventing a coronavirus infection, the method comprising providing to a subject an effective amount of a composition comprising a lipid vesicle comprising a therapeutic protein and an anti-viral therapeutic. In some embodiments, the anti-viral therapeutic is a nucleic acid, such as a small interfering RNA (siRNA), a small hairpin RNA (shRNA), or an antisense oligonucleotide, and the anti-viral nucleic acid is capable of targeting and reducing expression of a host protein and/or a viral protein. In some embodiments, the host protein is ACE2, TMPRSS2, 3CLpro, ALpro, or AT2. In some embodiments, the anti-viral nucleic acid is capable of targeting and reducing expression of ACE2. In some embodiments, the viral protein is a spike protein, an envelope protein, a membrane glycoprotein, a nucleocapsid protein, an RNA-dependent RNA polymerase, a replicase, or a helicase. In other embodiments, the anti-viral therapeutic is an anti-viral compound, and the anti-viral compound is an RNA polymerase inhibitor, a replicase inhibitor, or a helicase inhibitor.

In some embodiments, therapy provided herein comprises administration of a combination of therapeutic agents, such as lipid vesicles, liposomes, or exosomes; cells comprising lipid vesicles, liposomes, or exosomes; vaccine compositions comprising lipid vesicles, liposomes, or exosomes; or vaccine compositions comprising cells comprising lipid vesicles, liposomes, or exosomes, wherein the lipid vesicles, liposomes, or exosomes may be engineered to overexpress host or viral proteins. In some embodiments, the additional therapeutic comprises an agent for treating a viral infection, for example, a SARS-CoV-2 infection, including but not limited to steroids, zinc, vitamin C, Remdesivir, Tocilizumab, Anakinra, Beclomethasone, Betamethasone, Budesonide Cortisone, Dexamethasone, Hydrocortisone, Methylprednisolone, Prednisolone, Prednisone, Triamcinolone, Azithromycin, AC-55541, Apicidin, AZ3451, AZ8838, Bafilomycin A1, CCT 365623, Daunorubicin, E-52862, Entacapone, GB110, H-89, Haloperidol, Indomethacin, JQ1, Loratadine, Merimepodib, Metformin, Midostaurin, Migalastat, Mycophenolic acid, PB28, PD-144418, Ponatinib, Ribavirin, RS-PPCC, Ruxolitinib, RVX-208, S-verapamil, Silmitasertib, TMCB, UCPH-101, Valproic Acid, XL413, ZINC1775962367, ZINC4326719, ZINC4511851, ZINC95559591, 4E2RCat, ABBV-744, Camostat, Captopril, CB5083, Chloramphenicol, Chloroquine, Hydroxychloroquine, CPI-0610, Dabrafenib, DBeQ, dBET6, IHVR-19029, Linezolid, Lisinopril, Minoxidil, ML240, MZ1, Nafamostat, Pevonedistat, PS3061, Rapamycin (Sirolimus), Sanglifehrin A, Sapanisertib (INK128/M1N128), FK-506 (Tacrolimus), Ternatin 4 (DA3), Tigecycline, Tomivosertib (eFT-508), Verdinexor, WDB002, Zotatifin (eFT226), or a combination thereof.

A. Therapeutic Compositions

Aspects of the present disclosure comprise lipid vesicles. A lipid vesicle may be an artificial lipid vesicle (e.g., a liposome). Examples of artificial vesicles include multilamellar vesicles and unilamellar vesicles. A lipid vesicle may be a vesicle obtained or derived from a cell (e.g., an exosome). In some embodiments, a lipid vesicle of the present disclosure is an exosome. A cell may be engineered to produce exosomes of the present disclosure, including exosomes expressing one or more therapeutic proteins. In some embodiments, cells engineered to produce such exosomes are mesenchymal stem cells. In some embodiments, cells engineered to produce such exosomes are cells from a mammalian cell culture (e.g., 293T cells or 293F cells).

In some aspects, the disclosed lipid vesicles comprise one or more therapeutic proteins. A therapeutic protein, as disclosed herein, describes a protein capable of directly or indirectly facilitating treatment or prevention of a coronavirus infection. A therapeutic protein may comprise a transmembrane domain. A transmembrane domain of a therapeutic protein may be used to insert the protein into a lipid (e.g., phospholipid) region of a lipid vesicle. A therapeutic protein may comprise an external domain. An external domain, as described herein, refers to a protein domain which is expressed or provided external to a lipid membrane, such as on an exterior surface of a cell or lipid vesicle. An external domain may also be on an interior surface of a lipid vesicle.

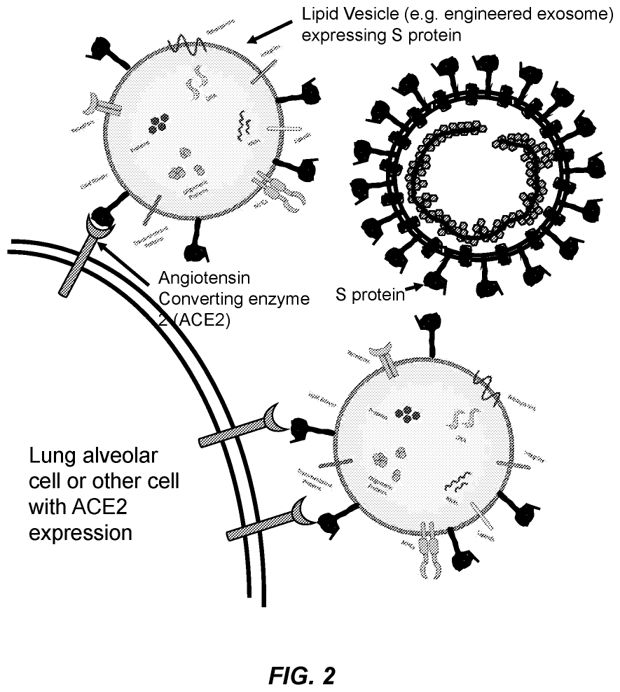

In some embodiments, a therapeutic protein is a coronavirus spike protein (also “S protein” or “spike glycoprotein” or “S glycoprotein”). In some embodiments, a coronavirus spike protein is a betacoronavirus spike protein. In some embodiments, a coronavirus spike protein is a SARS-CoV spike protein. In some embodiments, a coronavirus spike protein is a SARS-CoV-2 spike protein. FIG. 2 shows a schematic of lipid vesicles expressing a SARS-CoV-2 S protein, or portion thereof, on their surface. In this example, the lipid vesicles bind to the ACE2 protein on a lung alveolar cell, thereby preventing binding of the SARS-CoV-2 virus to the lung alveolar cell.

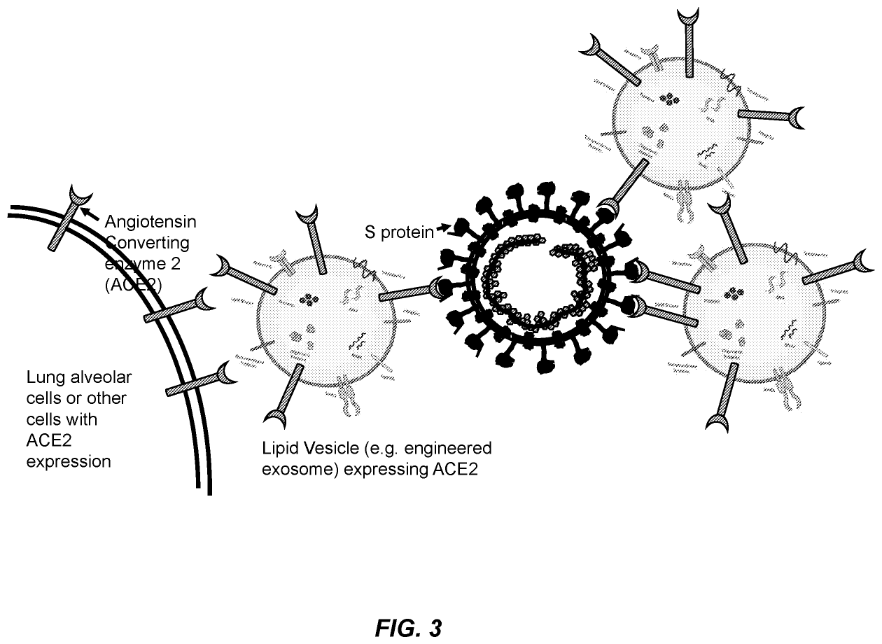

In some embodiments, a therapeutic protein is a protein capable of facilitating entry of a coronavirus into a cell. Examples of coronavirus entry proteins include integrins, Aminopeptidase N, carcinoembryonic-antigen-related cell-adhesion 1 (CEACAM1), and ACE2. In some embodiments, a therapeutic protein is ACE2. FIG. 3 shows a schematic of lipid vesicles expressing an ACE2 protein, or portion thereof, on their surface. In this example, the lipid vesicles bind to a SARS-CoV-2 coronavirus particle, functioning as a decoy receptor and preventing binding of the viral particles to lung alveolar cells.

In some aspects, the disclosed lipid vesicles comprise one or more anti-viral therapeutics. In such cases, lipid vesicles may be useful for delivery of an anti-viral therapeutic to a cell of interest. For example, a lipid vesicle comprising a coronavirus spike protein may be used for delivery of an anti-viral molecule to a virally infected cell. In some embodiments, an anti-viral therapeutic is a nucleic acid. Examples of anti-viral nucleic acids include small interfering RNA (siRNA), small hairpin RNA (shRNA), and antisense oligonucleotides. An anti-viral nucleic acid may be configured to target and reduce the expression of a host protein. For example, an anti-viral nucleic acid may be configured to target and reduce the expression of an ACE2 protein in a cell, thereby preventing the infection of the cell with a SARS-CoV-2 coronavirus. Further examples of host proteins which may be targeted include TMPRSS2 and AT2. A host protein may be any host protein such that reduction or elimination of expression of the host protein results in destruction of the host cell (e.g., proteins essential for cellular processes such as protein synthesis, endosomal transport, golgi function, and/or vesicular transport). An anti-viral nucleic acid may be configured to target and reduce the expression of a viral protein. An anti-viral nucleic acid may be configured to target any viral protein including, for example, a spike protein, an envelope protein, a membrane glycoprotein, a nucleocapsid protein, an RNA-dependent RNA polymerase, a replicase, or a helicase. Specific examples of viral proteins which may be targeted include nucleotprotein N, 3CL^{pro} and AL^{pro}. In some embodiments, an anti-viral therapeutic is an anti-viral compound (e.g., small molecule). An anti-viral compound may be an inhibitor of a viral enzyme. An anti-viral compound may be, for example, an RNA polymerase inhibitor, a replicase inhibitor, or a helicase inhibitor.

Embodiments of the disclosure relate to lipid vesicles formulated for use as a vaccine. For example, in some embodiments, a lipid vesicle expressing a coronavirus spike protein is formulated for use as a vaccine to prevent or reduce a coronavirus infection. A lipid vesicle of the present disclosure may be formulated with one or more adjuvants. Various vaccine adjuvants are known in the art and include, for example, aluminum and lipid-based adjuvants.

1. Inhibitory Oligonucleotides

In some aspects, the disclosure relates to inhibitory oligonucleotides that inhibit the gene expression of a viral entry protein, for example ACE2. Examples of an inhibitory oligonucleotides include but are not limited to siRNA (small interfering RNA), short hairpin RNA (shRNA), double-stranded RNA, an antisense oligonucleotide, a ribozyme, and an oligonucleotide encoding any thereof. An inhibitory oligonucleotide may inhibit the transcription of a gene or prevent the translation of a gene transcript in a cell. An inhibitory oligonucleotide acid may be from 16 to 1000 nucleotides long, and in certain embodiments from 18 to 100 nucleotides long. The oligonucleotide may have at least or may have at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 40, 50, 60, 70, 80, or 90 (or any range or value derivable therein) nucleotides. The oligonucleotide may be DNA, RNA, or a cDNA that encodes an inhibitory RNA.

As used herein, “isolated” means altered or removed from the natural state through human intervention. For example, an siRNA naturally present in a living animal is not “isolated,” but a synthetic siRNA, or an siRNA partially or completely separated from the coexisting materials of its natural state is “isolated.” An isolated siRNA can exist in substantially purified form, or can exist in a non-native environment such as, for example, a cell into which the siRNA has been delivered or a lipid vesicle into which the siRNA has been encapsulated.

Inhibitory oligonucleotides are well known in the art. For example, siRNA and double-stranded RNA have been described in U.S. Pat. Nos. 6,506,559 and 6,573,099, as well as in U.S. Patent Publications 2003/0051263, 2003/0055020, 2004/0265839, 2002/0168707, 2003/0159161, and 2004/0064842, all of which are herein incorporated by reference in their entirety.

Particularly, an inhibitory oligonucleotide may be capable of decreasing the expression of a viral entry protein, for example ACE2, by at least 10%, 20%, 30%, 40%, 50%, 60%, 70%, 75%, 80%, 90%, 95%, 99%, or more, or any range or value in between the foregoing.

In further embodiments, there are synthetic oligonucleotides that are viral entry protein (e.g., ACE2) inhibitors. An inhibitor may be between 17 to 25 nucleotides in length and comprises a 5′ to 3′ sequence that is at least 90% complementary to the 5′ to 3′ sequence of a mature viral entry protein (e.g., ACE2) mRNA. In certain embodiments, an inhibitor molecule is 17, 18, 19, 20, 21, 22, 23, 24, or 25 nucleotides in length, or any range derivable therein. Moreover, an inhibitor molecule has a sequence (from 5′ to 3′) that is or is at least 90, 91, 92, 93, 94, 95, 96, 97, 98, 99, 99.1, 99.2, 99.3, 99.4, 99.5, 99.6, 99.7, 99.8, 99.9 or 100% complementary, or any range derivable therein, to the 5′ to 3′ sequence of a mature viral entry protein (e.g., ACE2) mRNA, particularly a mature, naturally occurring mRNA. One of skill in the art could use a portion of the probe sequence that is complementary to the sequence of a mature mRNA as the sequence for an mRNA inhibitor. Moreover, that portion of the probe sequence can be altered so that it is still 90% complementary to the sequence of a mature mRNA.

In some embodiments, the inhibitory oligonucleotide is an analog and may include modifications, particularly modifications that increase nuclease resistance, improve binding affinity, and/or improve binding specificity. For example, when the sugar portion of a nucleoside or nucleotide is replaced by a carbocyclic moiety, it is no longer a sugar. Moreover, when other substitutions, such a substitution for the inter-sugar phosphodiester linkage are made, the resulting material is no longer a true species. All such compounds are considered to be analogs. Throughout this specification, reference to the sugar portion of a nucleic acid species shall be understood to refer to either a true sugar or to a species taking the structural place of the sugar of wild type nucleic acids. Moreover, reference to inter-sugar linkages shall be taken to include moieties serving to join the sugar or sugar analog portions in the fashion of wild type nucleic acids.

The present disclosure concerns modified oligonucleotides, i.e., oligonucleotide analogs or oligonucleosides, and methods for effecting the modifications. These modified oligonucleotides and oligonucleotide analogs may exhibit increased chemical and/or enzymatic stability relative to their naturally occurring counterparts. Extracellular and intracellular nucleases generally do not recognize and therefore do not bind to the backbone-modified compounds. When present as the protonated acid form, the lack of a negatively charged backbone may facilitate cellular penetration.

The modified internucleoside linkages are intended to replace naturally-occurring phosphodiester-5′-methylene linkages with four atom linking groups to confer nuclease resistance and enhanced cellular uptake to the resulting compound.

Modifications may be achieved using solid supports which may be manually manipulated or used in conjunction with a DNA synthesizer using methodology commonly known to those skilled in DNA synthesizer art. Generally, the procedure involves functionalizing the sugar moieties of two nucleosides which will be adjacent to one another in the selected sequence. In a 5′ to 3′ sense, an “upstream” synthon such as structure H is modified at its terminal 3′ site, while a “downstream” synthon such as structure H1 is modified at its terminal 5′ site.

Oligonucleosides linked by hydrazines, hydroxylarnines, and other linking groups can be protected by a dimethoxytrityl group at the 5′-hydroxyl and activated for coupling at the 3′-hydroxyl with cyanoethyldiisopropyl-phosphite moieties. These compounds can be inserted into any desired sequence by standard, solid phase, automated DNA synthesis techniques. One of the most popular processes is the phosphoramidite technique. Oligonucleotides containing a uniform backbone linkage can be synthesized by use of CPG-solid support and standard nucleic acid synthesizing machines such as Applied Biosystems Inc. 380B and 394 and Milligen/Biosearch 7500 and 8800s. The initial nucleotide (number 1 at the 3′-terminus) is attached to a solid support such as controlled pore glass. In sequence specific order, each new nucleotide is attached either by manual manipulation or by the automated synthesizer system.

Free amino groups can be alkylated with, for example, acetone and sodium cyanoboro hydride in acetic acid. The alkylation step can be used to introduce other, useful, functional molecules on the macromolecule. Such useful functional molecules include but are not limited to reporter molecules, RNA cleaving groups, groups for improving the pharmacokinetic properties of an oligonucleotide, and groups for improving the pharmacodynamic properties of an oligonucleotide. Such molecules can be attached to or conjugated to the macromolecule via attachment to the nitrogen atom in the backbone linkage. Alternatively, such molecules can be attached to pendent groups extending from a hydroxyl group of the sugar moiety of one or more of the nucleotides. Examples of such other useful functional groups are provided by WO1993007883, which is herein incorporated by reference, and in other of the above-referenced patent applications.

Solid supports may include any of those known in the art for polynucleotide synthesis, including controlled pore glass (CPG), oxalyl controlled pore glass, TentaGel Support—an aminopolyethyleneglycol derivatized support or Poros—a copolymer of polystyrene/divinylbenzene. Attachment and cleavage of nucleotides and oligonucleotides can be effected via standard procedures. As used herein, the term solid support further includes any linkers (e.g., long chain alkyl amines and succinyl residues) used to bind a growing oligonucleoside to a stationary phase such as CPG. In some embodiments, the oligonucleotide may be further defined as having one or more locked nucleotides, ethylene bridged nucleotides, peptide nucleic acids, or a 5′(E)-vinyl-phosphonate (VP) modification. In some embodiments, the oligonucleotides has one or more phosphorothioated DNA or RNA bases.

B. Cellular Therapies

Aspects of the disclosure include cellular therapies. In some embodiments, cellular therapies comprise cells engineered to generate lipid vesicles comprising one or more therapeutic proteins, as disclosed elsewhere herein. In some embodiments, mesenchymal stem cells are engineered to generate exosomes expressing one or more therapeutic proteins (e.g., SARS-CoV-2 spike protein, ACE2) and provided to a subject to treat or prevent a coronavirus infection. The mesenchymal stem cells may be from the subject, or from a different subject.

1. Cell Culture

In some embodiments, cells may be cultured for at least between about 10 days and about 40 days, for at least between about 15 days and about 35 days, for at least between about 15 days and 21 days, such as for at least about 15, 16, 17, 18, 19 or 21 days. In some embodiments, the cells of the disclosure may be cultured for no longer than 60 days, or no longer than 50 days, or no longer than 45 days. The cells may be cultured for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 days. The cells may be cultured in the presence of a liquid culture medium. Typically, the medium may comprise a basal medium formulation as known in the art. Many basal media formulations can be used to culture cells herein, including but not limited to Eagle's Minimum Essential Medium (MEM), Dulbecco's Modified Eagle's Medium (DMEM), alpha modified Minimum Essential Medium (alpha-MEM), Basal Medium Essential (BME), Iscove's Modified Dulbecco's Medium (IMDM), BGJb medium, F-12 Nutrient Mixture (Ham), Liebovitz L-15, DMEM/F-12, Essential Modified Eagle's Medium (EMEM), RPMI-1640, and modifications and/or combinations thereof. Compositions of the above basal media are generally known in the art, and it is within the skill of one in the art to modify or modulate concentrations of media and/or media supplements as necessary for the cells cultured. In some embodiments, a culture medium formulation may be explants medium (CEM) which is composed of IMDM supplemented with 10% fetal bovine serum (FBS), 100 U/ml penicillin G, 100 μg/ml streptomycin and 2 mmol/L-glutamine. Other embodiments may employ further basal media formulations, such as chosen from the ones above.

Any medium capable of supporting cells in vitro may be used to culture the cells. Media formulations that can support the growth of cells include, but are not limited to, Dulbecco's Modified Eagle's Medium (DMEM), alpha modified Minimal Essential Medium (αMEM), and Roswell Park Memorial Institute Media 1640 (RPMI Media 1640) and the like. Typically, up to 20% fetal bovine serum (FBS) or 1-20% horse serum is added to the above medium in order to support the growth of cells. A defined medium, however, also can be used if the growth factors, cytokines, and hormones necessary for culturing cells are provided at appropriate concentrations in the medium. Media useful in the methods of the disclosure may comprise one or more compounds of interest, including, but not limited to, antibiotics, mitogenic compounds, or differentiation compounds useful for the culturing of cells. The cells may be grown at temperatures between 27° C. to 40° C., such as 31° C. to 37° C., and may be in a humidified incubator. The carbon dioxide content may be maintained between 2% to 10% and the oxygen content may be maintained between 1% and 22%. The disclosure, however, should in no way be construed to be limited to any one method of isolating and culturing cells. Rather, any method of isolating and culturing cells should be construed to be included in the present disclosure.

For use in the cell culture, media can be supplied with one or more further components. For example, additional supplements can be used to supply the cells with the necessary trace elements and substances for optimal growth and expansion. Such supplements include insulin, transferrin, selenium salts, and combinations thereof. These components can be included in a salt solution such as, but not limited to, Hanks' Balanced Salt Solution (HBSS), Earle's Salt Solution. Further antioxidant supplements may be added, e.g., P-mercaptoethanol. While many media already contain amino acids, some amino acids may be supplemented later, e.g., L-glutamine, which is known to be less stable when in solution. A medium may be further supplied with antibiotic and/or antimycotic compounds, such as, typically, mixtures of penicillin and streptomycin, and/or other compounds, exemplified but not limited to, amphotericin, ampicillin, gentamicin, bleomycin, hygromycin, kanamycin, mitomycin, mycophenolic acid, nalidixic acid, neomycin, nystatin, paromomycin, polymyxin, puromycin, rifampicin, spectinomycin, tetracycline, tylosin, and zeocin. Also contemplated is supplementation of cell culture medium with mammalian plasma or sera. Plasma or sera often contain cellular factors and components that are necessary for viability and expansion. The use of suitable serum replacements is also contemplated.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed. In particular embodiments, cells are cultured in a cell culture system comprising a cell culture medium, preferably in a culture vessel, in particular a cell culture medium supplemented with a substance suitable and determined for protecting the cells from in vitro aging and/or inducing in an unspecific or specific reprogramming.

### III. Administration of Therapeutic Compositions

Embodiments of the disclosure relate to compositions and methods comprising therapeutic compositions, including lipid vesicles, liposomes, or exosomes, which may comprise therapeutic proteins and/or anti-viral therapeutics.

In some embodiments, the therapy comprises lipid vesicles, which may comprise therapeutic proteins and/or anti-viral therapeutics. In some embodiments, the therapy comprises liposomes, which may comprise therapeutic proteins and/or anti-viral therapeutics. In some embodiments, the therapy comprises exosomes, which may comprise therapeutic proteins and/or anti-viral therapeutics. In some embodiments, the therapy comprises cells configured to produce lipid vesicles, which may comprise therapeutic proteins and/or anti-viral therapeutics. In some embodiments, the therapy comprises cells configured to produce liposomes, which may comprise therapeutic proteins and/or anti-viral therapeutics. In some embodiments, the therapy comprises cells configured to produce exosomes, which may comprise therapeutic proteins and/or anti-viral therapeutics. In some embodiments, the therapy comprises vaccine compositions comprising lipid vesicles, which may comprise therapeutic proteins and/or anti-viral therapeutics. In some embodiments, the therapy comprises vaccine compositions comprising liposomes, which may comprise therapeutic proteins and/or anti-viral therapeutics. In some embodiments, the therapy comprises vaccine compositions comprising exosomes, which may comprise therapeutic proteins and/or anti-viral therapeutics. In some embodiments, the therapy comprises vaccine compositions comprising cells configured to produce lipid vesicles, which may comprise therapeutic proteins and/or anti-viral therapeutics. In some embodiments, the therapy comprises vaccine compositions comprising cells configured to produce liposomes, which may comprise therapeutic proteins and/or anti-viral therapeutics. In some embodiments, the therapy comprises vaccine compositions comprising cells configured to produce exosomes, which may comprise therapeutic proteins and/or anti-viral therapeutics. Any of these disease therapies may be excluded. Combinations of these therapies may also be administered.

The therapy provided herein may comprise administration of a combination of therapeutic compositions, such as a first disease therapy (e.g., lipid vesicles, which may comprise therapeutic proteins and/or anti-viral therapeutics) and one or more additional disease therapies (e.g., anti-viral therapeutics). The therapies may be administered in any suitable manner known in the art. For example, the therapies may be administered sequentially (at different times) or concurrently (at the same time or approximately the same time; also “simultaneously” or “substantially simultaneously”). Different therapies may be administered in one composition or in more than one composition, such as 2 compositions, 3 compositions, or 4 compositions. Various combinations of the agents may be employed.

In some embodiments, the compositions comprising lipid vesicles, liposomes, or exosomes, which may comprise therapeutic proteins and/or anti-viral therapeutics, are delivered to the subject a single time. In some embodiments, the compositions comprising lipid vesicles, liposomes, or exosomes, which may comprise therapeutic proteins and/or anti-viral therapeutics, are delivered to the subject multiple times, such as once a day, more than once a day, once a week, more than once a week, once a month, more than once a month, once a year, or more than once a year. In some embodiments, lipid vesicles, liposomes, or exosomes, which may comprise therapeutic proteins and/or anti-viral therapeutics, are administered to the subject multiple times. In some embodiments, lipid vesicles, liposomes, or exosomes, which may comprise therapeutic proteins and/or anti-viral therapeutics, are administered to the subject a single time. Multiple treatments may or may not have the same formulations and/or routes of administration(s).

In some embodiments, the compositions comprising lipid vesicles, liposomes, or exosomes, which may comprise therapeutic proteins and/or anti-viral therapeutics, are delivered after onset of a disease, for example, a coronavirus infection. In some embodiments, the compositions comprising lipid vesicles, liposomes, or exosomes, which may comprise therapeutic proteins and/or anti-viral therapeutics, are delivered before onset of a disease, for example, a coronavirus infection.

The therapeutic agents of the disclosure may be administered by the same route of administration or by different routes of administration. In some embodiments, the therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

In some embodiments, the composition(s) may be administered via a parenteral route. As used herein, the term “parenteral” includes routes that bypass the alimentary tract. Specifically, the pharmaceutical compositions disclosed herein may be administered for example, but not limited to retro-orbitally, intracerebrally, intracranially, intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (see, e.g., U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions may be prepared by incorporating the active compounds in the required amount in the appropriate solvent with various of the other ingredients enumerated above, as required, followed by filtered sterilization, for example. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

In other embodiments, the pharmaceutical composition(s) may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (see, e.g., Takenaga et al., 1998) and lysophosphatidyl-glycerol compounds (see, e.g., U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in, e.g., U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present disclosure for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### IV. Kits

Certain aspects of the present invention also concern kits containing compositions of the invention or compositions to implement methods of the invention. In some embodiments, kits can be used to neutralize coronavirus in a subject or a sample. In certain embodiments, a kit contains, contains at least or contains at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 100, 500, 1,000 or more probes, primers or primer sets, synthetic molecules or inhibitors, or any value or range and combination derivable therein. In some embodiments, a kit contains one or more lipid vesicles comprising therapeutic protein, or portion or variant thereof, configured to bind to one or more coronavirus spike proteins, including the lipid vesicles disclosed herein. In some embodiments, a kit contains one or more lipid vesicles comprising an ACE2 protein, or portion or variant thereof. For example, a kit may comprise at least 1, 2, 3, 4, 5, 6, 7, 8, 9, 10 or more lipid vesicles disclosed herein that interact with and neutralize a coronavirus spike protein.

Kits may comprise components, which may be individually packaged or placed in a container, such as a tube, bottle, vial, syringe, or other suitable container means.

Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

Kits for using probes, synthetic nucleic acids, nonsynthetic nucleic acids, and/or inhibitors of the disclosure for prognostic or diagnostic applications are included as part of the disclosure. Specifically contemplated are any such molecules corresponding to any biomarker identified herein, which includes nucleic acid primers/primer sets and probes that are identical to or complementary to all or part of a biomarker, which may include noncoding sequences of the biomarker, as well as coding sequences of the biomarker.

Kits may further comprise instructions for use. For example, in some embodiments, a kit comprises instructions for detecting a coronavirus antibody in a subject or a sample. In some embodiments, a kit comprises instructions for neutralizing a coronavirus in a subject or a sample.

It is contemplated that any method or composition described herein can be implemented with respect to any other method or composition described herein and that different embodiments may be combined. The claims originally filed are contemplated to cover claims that are multiply dependent on any filed claim or combination of filed claims.

### EXAMPLES

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the invention.

### Example 1—Use of Exosomes Comprising Coronavirus Spike Protein as a Vaccine for Prevention of Coronavirus Infection

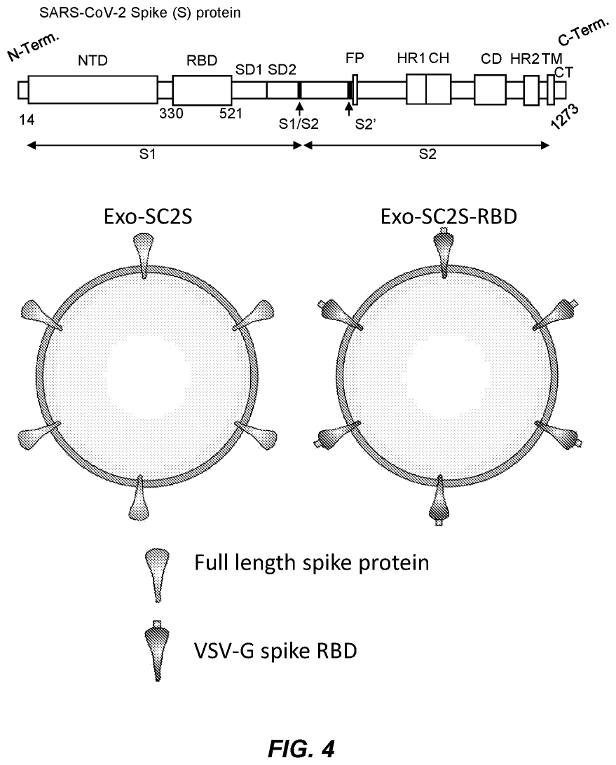

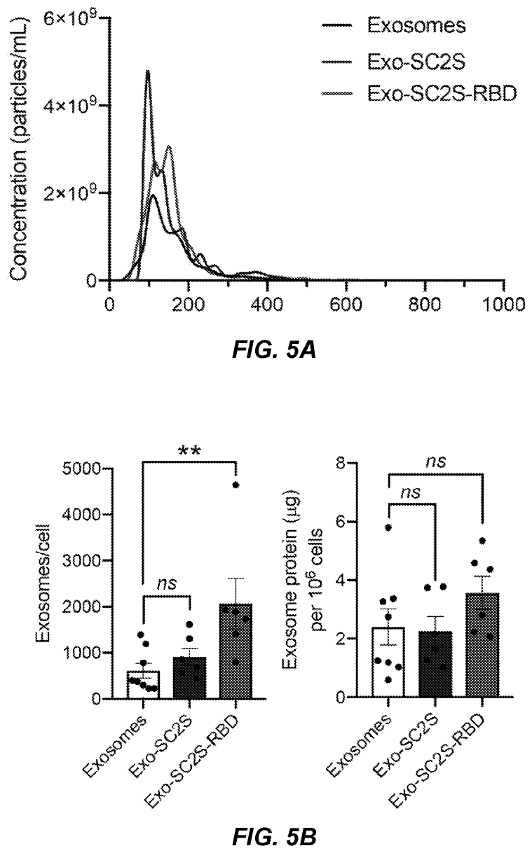

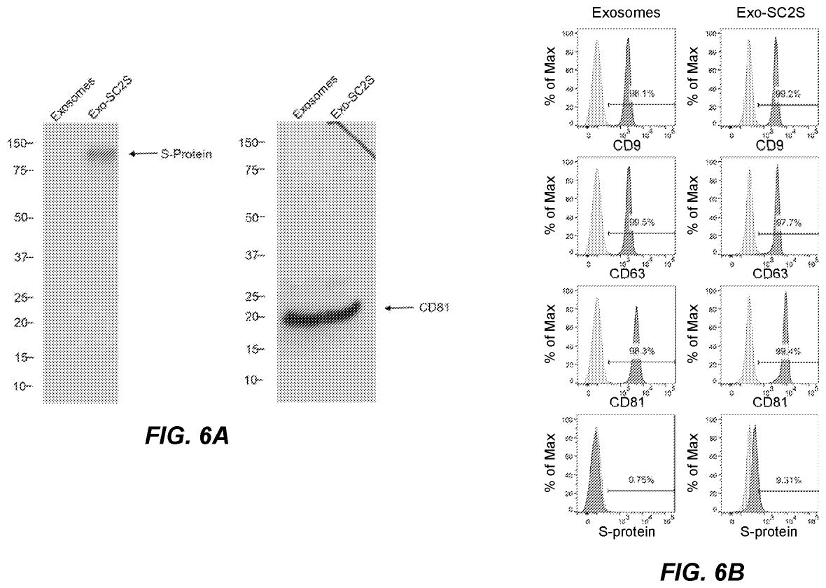

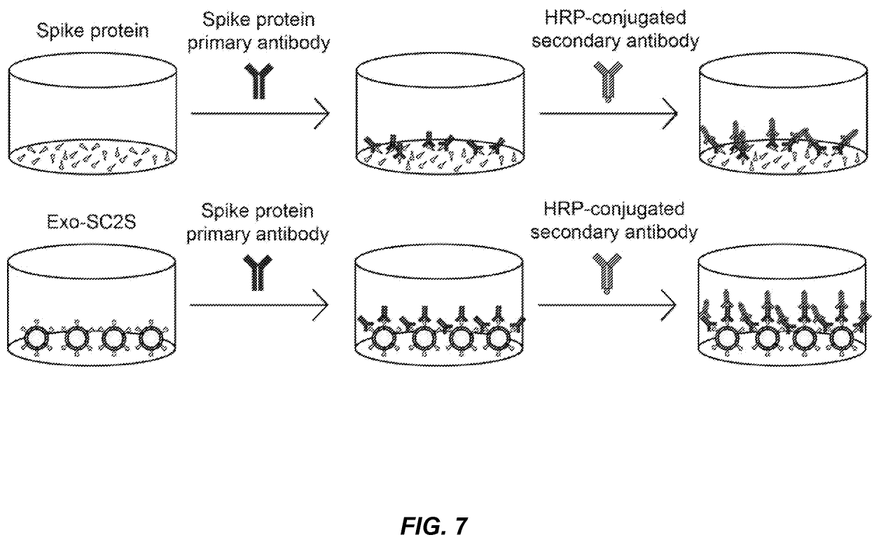

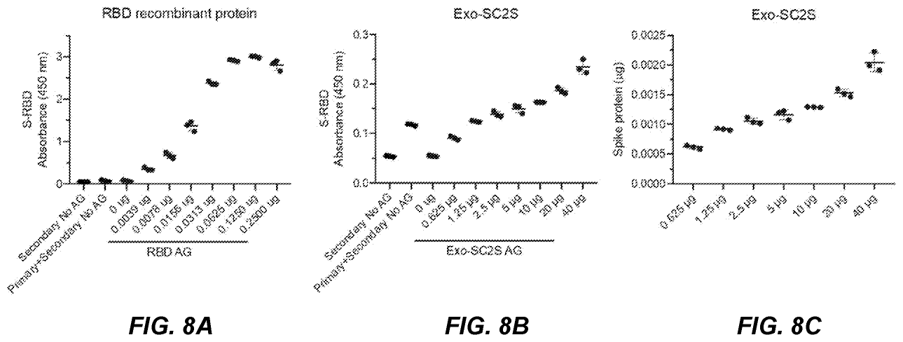

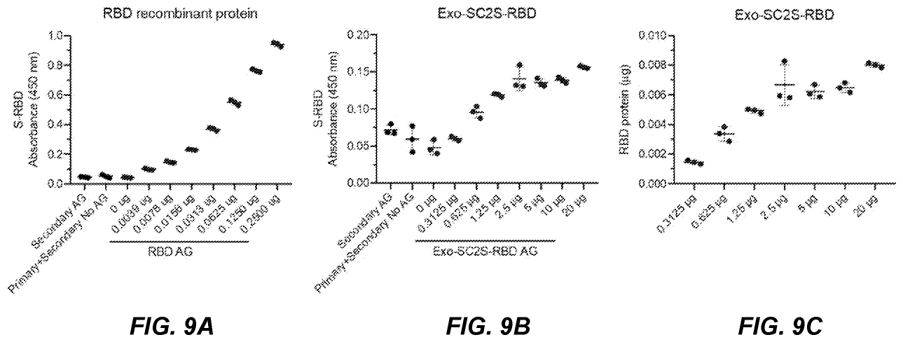

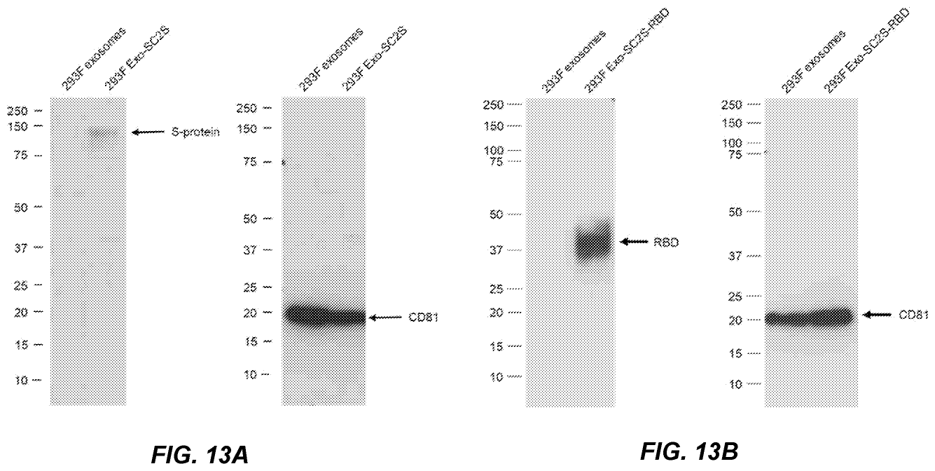

Exosomes similar in size to the SARS-CoV-2 virus and expressing SARS-CoV-2 spike protein (Exo-SC2S) or the RBD of the SARS-CoV-2 spike protein (Exo-SC2S-RBD) on their surface were generated (FIG. 4, FIG. 5) by 293F cells (FIG. 13). The exosomes were isolated from the culture media, and S-protein expression was confirmed by Western blot (FIG. 6A) and by flow cytometry (FIG. 6B). ELISA assays (FIG. 7) were used to quantify the levels of S-protein (Exo-SC2S exosomes, FIG. 8) or S-protein RBD (Exo-SC2S-RBD, FIG. 9) expressed by Exo-SC2S and Exo-SC2S-RBD exosomes, respectively. Results from these ELISA assays demonstrate the specificity of the Exo-SC2S and Exo-SC2S-RBD exosomes for the SARS-CoV-2 RBD.

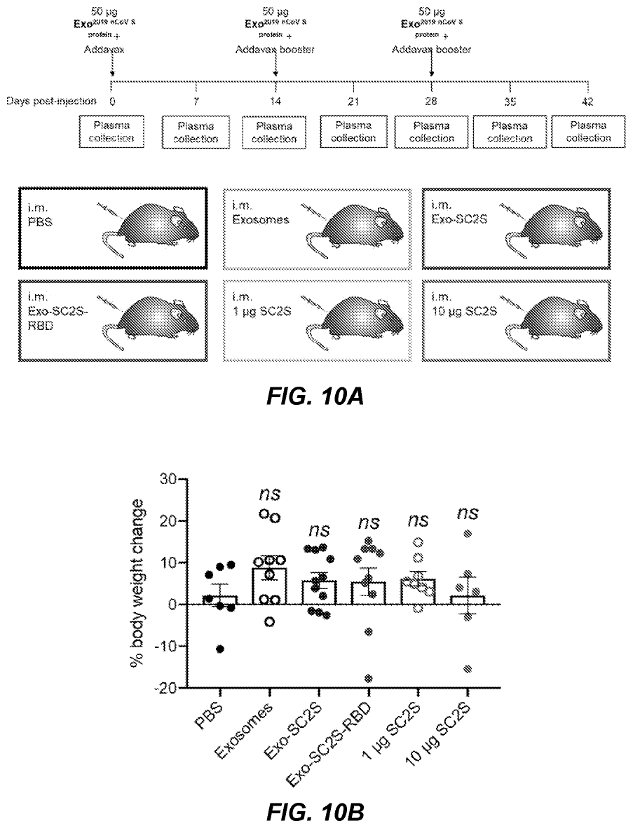

These Exo-SC2S and Exo-SC2S-RBD exosomes (50 μg) were formulated into a vaccine composition (FIG. 10A), the vaccine composition also comprising the oil-in-water nano-emulsion vaccine adjuvant Addavax. Vaccine compositions comprising PBS, exosomes lacking S-protein expression (exosomes), and S-protein (SC2S) were also administered as controls (FIG. 10A). The vaccine composition was intramuscularly administered to mice three times on Days 0, 14, and 28 (FIG. 10A). Administration of the vaccine compositions did not result in a significant change in percent body weight (FIG. 10B).

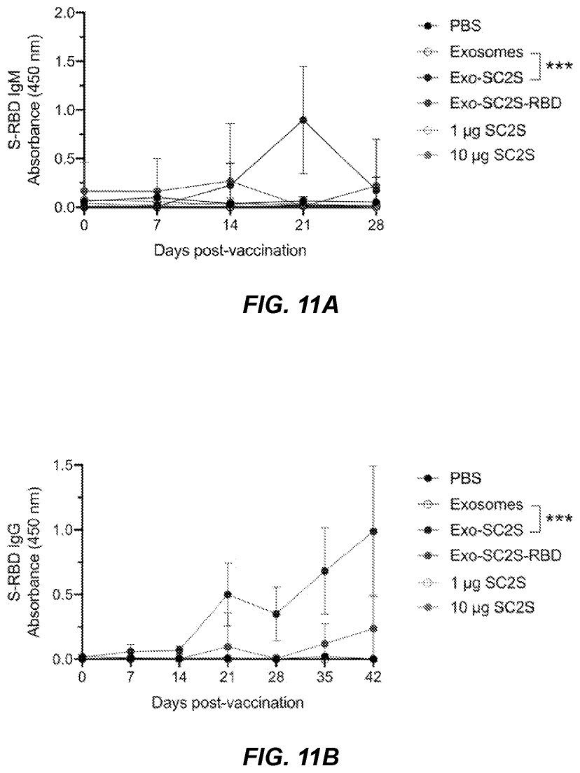

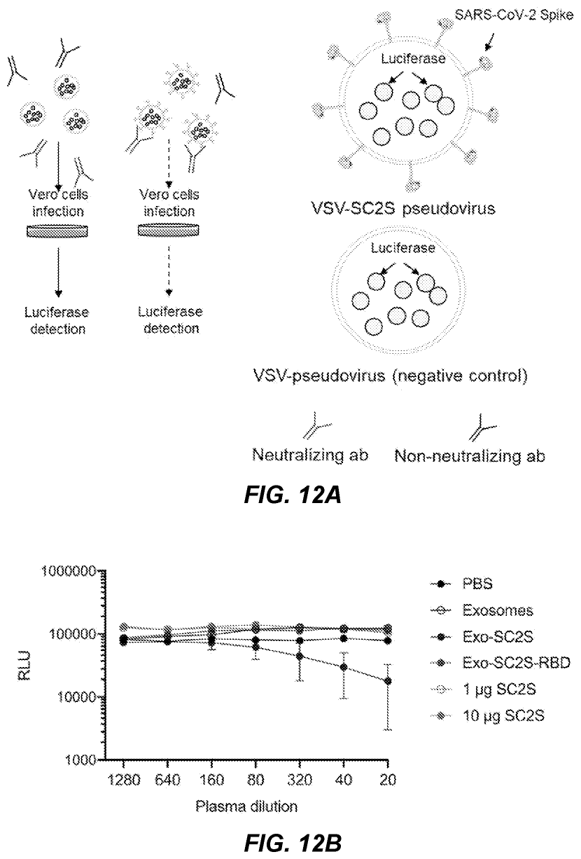

Following intramuscular vaccination with vaccine compositions comprising either Exo-SC2S and Exo-SC2S-RBD exosomes, IgM (FIG. 11A) and IgG (FIG. 11B) antibodies against SARS-CoV-2 spike protein RBD were generated in the mice, with the Exo-SC2S exosome vaccine composition resulting in significantly greater antibody production as compared to the vaccine compositions comprising exosomes lacking S-protein expression. The inventors next sought to determine whether the antibodies produced following vaccination with the Exo-SC2S and Exo-SC2S-RBD exosome vaccine compositions were neutralizing against the SARS-CoV-2 virus. Using either a VSV-pseudovirus expressing SARS-CoV-2 S-protein on its surface or VSV-only pseudovirus (negative control) (FIG. 12A), the inventors showed that the antibodies in the blood of mice following Exo-SC2S intramuscular vaccination exhibited neutralizing activity (FIG. 12B). These vaccine composition can also be provided to human subjects at risk for infection with SARS-CoV-2, thereby generating antibodies in the subject against the SARS-CoV-2 spike protein.

### Example 2—Use of Exosomes Comprising Coronavirus Spike Protein to Reduce ACE2 Expression in Lung Alveolar Cells

293T cells are engineered to generate exosomes expressing SARS-CoV-2 spike protein or the RBD of the SARS-CoV-2 spike protein on their surface and to internally contain an siRNA targeting angiotensin converting enzyme 2 (ACE2). Exosomes with similar size as the SARS-CoV-2 virus are isolated from the culture media. Such engineered exosomes (Exo^{SARS-CoV-2 S protein}) are formulated into a pharmaceutical composition. The pharmaceutical composition is provided intranasally to a subject having a SARS-CoV-2 infection, thereby delivering the siRNA to ACE2-expressing lung alveolar cells and reducing ACE2 expression in these cells.

### Example 3—Use of Exosomes Comprising ACE2 as a Therapeutic for Treatment of Coronavirus Infection

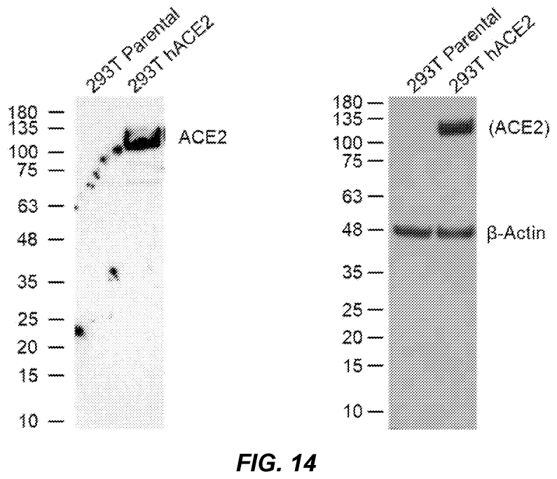

293T cells were engineered to generate exosomes similar size as the SARS-CoV-2 virus and expressing ACE2 on their surface (FIG. 14). The exosomes were isolated from the culture media, and ACE2 protein expression was confirmed by Western blot (FIG. 14). Such engineered exosomes (Exo^{ACE2}) may be formulated into a pharmaceutically acceptable therapeutic composition.

### Example 4—Use of Mesenchymal Stem Cells Engineered to Generate Exosomes Comprising Coronavirus Spike Protein as a Therapeutic for Treatment of Coronavirus Infection

Mesenchymal stem cells are engineered to generate exosomes expressing SARS-CoV-2 spike protein or the RBD of the SARS-CoV-2 spike protein on their surface. The exosomes are isolated and formulated into a pharmaceutically acceptable therapeutic composition. The therapeutic composition is provided to a subject having a SARS-CoV-2 infection, thereby reducing the severity of the symptoms of the infection.

### Example 5—Use of 293F Cells Engineered to Generate Exosomes Comprising Coronavirus Spike Protein as a Therapeutic for Treatment of Coronavirus Infection

293F cells are engineered to generate exosomes expressing SARS-CoV-2 spike protein or the RBD of the SARS-CoV-2 spike protein on their surface. The exosomes are isolated and formulated into a pharmaceutically acceptable therapeutic composition. The therapeutic composition is provided to a subject having a SARS-CoV-2 infection, thereby reducing the severity of the symptoms of the infection.

### Example 6—Use of Mesenchymal Stem Cells Engineered to Generate Exosomes Comprising Coronavirus Spike Protein to Reduce ACE2 Expression in Lung Alveolar Cells

Mesenchymal stem cells are engineered to generate exosomes expressing SARS-CoV-2 spike protein or the RBD of the SARS-CoV-2 spike protein on their surface and to internally contain an siRNA targeting angiotensin converting enzyme 2 (ACE2). The exosomes are isolated and formulated into a pharmaceutically acceptable therapeutic composition. The therapeutic composition is provided to a subject having a SARS-CoV-2 infection, thereby reducing the severity of the symptoms of the infection.

### Example 7—Use of 293T Cells Engineered to Generate Exosomes Comprising Coronavirus Spike Protein to Reduce ACE2 Expression in Lung Alveolar Cells

293T cells are engineered to generate exosomes expressing SARS-CoV-2 spike protein or the RBD of the SARS-CoV-2 spike protein on their surface and to internally contain an siRNA targeting angiotensin converting enzyme 2 (ACE2). The exosomes are isolated and formulated into a pharmaceutically acceptable therapeutic composition. The therapeutic composition is provided to a subject having a SARS-CoV-2 infection, thereby reducing the severity of the symptoms of the infection.

### Example 8—Use of Mesenchymal Stem Cells Engineered to Generate Exosomes Comprising ACE2 as a Therapeutic for Treatment of Coronavirus Infection

Mesenchymal stem cells are engineered to generate exosomes expressing ACE2 on their surface. The exosomes are isolated and formulated into a pharmaceutically acceptable therapeutic composition. The therapeutic composition is provided to a subject having a SARS-CoV-2 infection, thereby reducing the severity of the symptoms of the infection.

### Example 9—Use of 293T Cells Engineered to Generate Exosomes Comprising ACE2 as a Therapeutic for Treatment of Coronavirus Infection

293T cells are engineered to generate exosomes expressing ACE2 on their surface. The exosomes are isolated and formulated into a pharmaceutically acceptable therapeutic composition. The therapeutic composition is provided to a subject having a SARS-CoV-2 infection, thereby reducing the severity of the symptoms of the infection.

All of the methods disclosed and claimed herein can be made and executed without undue experimentation in light of the present disclosure. While the compositions and methods of this invention have been described in terms of certain embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A lipid vesicle comprising a SARS-CoV-2 spike protein, or portion or variant thereof, comprising a transmembrane domain and an external domain, wherein the external domain is on an exterior surface of the lipid vesicle.

2. The lipid vesicle of claim 1, wherein the external domain comprises an S1 domain.

3. The lipid vesicle of claim 1, wherein the external domain comprises an S2 domain.

4. The lipid vesicle of claim 1, wherein the external domain comprises an S1 domain and an S2 domain.

5. The lipid vesicle of any of claims 1-4, wherein the lipid vesicle is a liposome.

6. The lipid vesicle of any of claims 1-4, wherein the lipid vesicle is an exosome.

7. The lipid vesicle of any of claims 1-6, wherein the lipid vesicle further comprises an anti-viral therapeutic.

8. The lipid vesicle of claim 7, wherein the anti-viral therapeutic is a nucleic acid.

9. The lipid vesicle of claim 8, wherein the anti-viral nucleic acid is a small interfering RNA (siRNA), a small hairpin RNA (shRNA), or an antisense oligonucleotide.

10. The lipid vesicle of claim 8 or 9, wherein the anti-viral nucleic acid is configured to target and reduce expression of a host protein, wherein the host protein is ACE2, TMPRSS2, 3CLpro, ALpro, or AT2.

11. The method of claim 10, wherein the host protein is ACE2.

12. The lipid vesicle of claim 8 or 9, wherein the anti-viral nucleic acid is configured to target and reduce expression of a viral protein, wherein the viral protein is a spike protein, an envelope protein, a membrane glycoprotein, a nucleocapsid protein, an RNA-dependent RNA polymerase, a replicase, or a helicase.

13. The lipid vesicle of claim 7, wherein the anti-viral therapeutic is an anti-viral compound.

14. The lipid vesicle of claim 13, wherein the anti-viral compound is an RNA polymerase inhibitor, a replicase inhibitor, or a helicase inhibitor.

15. A cell configured to produce the lipid vesicle of any of claims 1-14.

16. The cell of claim 15, wherein the cell is a mesenchymal stem cell.

17. The cell of claim 15, wherein the cell is from a mammalian cell line.

18. The cell of claim 17, wherein the cell is a 293T cell.

19. The cell of claim 17, wherein the cell is a 293F cell.

20. A method for preventing or reducing a SARS-CoV-2 infection, the method comprising providing to a subject an effective amount of a composition comprising the lipid vesicle of any of claims 1-14 or the cell of any of claims 15-18.

21. The method of claim 20, wherein the subject is at risk for the SARS-CoV-2 infection.

22. The method of claim 20 or 21, wherein the composition further comprises a vaccine adjuvant.

23. The method of claim 22, wherein the vaccine adjuvant is aluminum or a lipid-based adjuvant.

24. The method of any of claims 20-23, wherein the composition is provided to the subject via intranasal administration.

25. The method of any of claims 20-23, wherein the composition is provided to the subject via intraperitoneal administration.

26. The method of any of claims 20-23, wherein the composition is provided to the subject via intramuscular administration.

27. The method of any of claims 20-26, wherein the subject is infected with the SARS-CoV-2.

28. A method for treating or preventing a coronavirus infection, the method comprising providing to a subject an effective amount of a composition comprising: a lipid vesicle comprising a therapeutic protein comprising a transmembrane domain and an external domain, wherein the external domain is on an exterior surface of the lipid vesicle.

29. The method of claim 28, wherein the coronavirus is a betacoronavirus.

30. The method of claim 29, wherein the coronavirus is SARS-CoV-2.

31. The method of any of claims 28-30, wherein the therapeutic protein is a coronavirus spike protein or portion or variant thereof.

32. The method of claim 31, wherein the external domain comprises an S1 domain

33. The method of claim 31, wherein the external domain comprises an S2 domain.

34. The method of claim 31, wherein the external domain comprises an S1 domain and an S2 domain.

35. The method of any of claims 28-30, wherein the therapeutic protein is an angiotensin converting enzyme 2 (ACE2) protein or portion or variant thereof.

36. The method of claim 35, wherein the external domain comprises a PD domain

37. The method of claim 35, wherein the external domain comprises a CLD domain.

38. The method of claim 35, wherein the external domain comprises a PD domain and a CLD domain.

39. The method of any of claims 28-38, wherein the subject is at risk for the coronavirus infection.

40. The method of claim 39, wherein the composition further comprises a vaccine adjuvant.

41. The method of claim 40, wherein the vaccine adjuvant is aluminum or a lipid-based adjuvant.

42. The method of any of claims 28-38, wherein the subject is infected with the coronavirus.

43. The method of any of claims 28-42, wherein the composition is provided to the subject via intranasal administration.

44. The method of any of claims 28-42, wherein the composition is provided to the subject via intraperitoneal administration.

45. The method of any of claims 28-42, wherein the composition is provided to the subject via intramuscular administration.

46. The method of any of claims 28-45, wherein the lipid vesicle is a liposome.

47. The method of any of claims 28-45, wherein the lipid vesicle is an exosome.

48. The method of any of claims 28-47, wherein the lipid vesicle further comprises an anti-viral therapeutic.

49. The method of claim 48, wherein the anti-viral therapeutic is a nucleic acid.

50. The method of claim 49, wherein the anti-viral nucleic acid is a small interfering RNA (siRNA), a small hairpin RNA (shRNA), or an antisense oligonucleotide.

51. The method of claim 49 or 50, wherein the anti-viral nucleic acid is capable of targeting and reducing expression of a host protein, wherein the host protein is ACE2, TMPRSS2, 3CLpro, ALpro, or AT2.

52. The method of claim 51, wherein the anti-viral nucleic acid is capable of targeting and reducing expression of ACE2.

53. The method of claim 49 or 50, wherein the anti-viral nucleic acid is capable of targeting and reducing expression of a viral protein, wherein the viral protein is a spike protein, an envelope protein, a membrane glycoprotein, a nucleocapsid protein, an RNA-dependent RNA polymerase, a replicase, or a helicase.

54. The method of claim 48, wherein the anti-viral therapeutic is an anti-viral compound.

55. The method of claim 54, wherein the anti-viral compound is an RNA polymerase inhibitor, a replicase inhibitor, or a helicase inhibitor.

56. The method of any of claims 28-55, wherein the method comprises providing a cell configured to produce the lipid vesicle.

57. The method of claim 56, wherein the cell is a mesenchymal stem cell.

58. The method of claim 56, wherein the cell is from a mammalian cell line.

59. The method of claim 58, wherein the cell is a 293T cell.

60. The method of claim 58, wherein the cell is a 293F cell.

61. A method for treating or preventing a SARS-CoV-2 infection, the method comprising providing to a subject an effective amount of a composition comprising a lipid vesicle comprising ACE2 or a portion or variant thereof.

62. The method of claim 61, wherein the lipid vesicle comprises a portion of ACE2.

63. The method of claim 62, wherein the portion of ACE2 comprises a PD domain.

64. The method of claim 62, wherein the portion of ACE2 comprises a CLD domain.

65. The method of claim 62, wherein the external domain comprises a PD domain and a CLD domain.

66. The method of claim 61, wherein the lipid vesicle comprises ACE2.

67. A method for treating a SARS-CoV-2 infection, the method comprising providing to a subject an effective amount of a composition comprising a lipid vesicle comprising a SARS-CoV-2 spike protein or portion or variant thereof.

68. The method of claim 67, wherein the subject has been diagnosed with a SARS-CoV-2 infection.

69. The method of claim 67 or 68, wherein the lipid vesicle comprises a portion of the SARS-CoV-2 spike protein.

70. The method of claim 69, wherein the portion of the SARS-CoV-2 spike protein comprises a RBD domain.
